(19) United States
(12) Patent Application Publication
(10) Pub. No.: US 2007/0099997 A1
(43) Pub. Date: May 3, 2007

(54) Potassium or sodium salt of (-)-2-{'2-(4-hydroxyphenyl) ethyl!-thio-3-'4-(2-{4-'(methylsulfonyl) oxy! phenoxy}ethyl) phenyl! propanoic acid and their use in medicine

(76) Inventors: Matti Ahlqvist, Molndal (SE); Martin Hans Bohlin, Sodertalje (SE)

Correspondence Address: MORGAN LEWIS & BOCKIUS LLP, 1111 PENNSYLVANIA AVENUE NW, WASHINGTON, DC, 20004, US

(21) Appl. No.: 10/561,170

(22) PCT Filed: Jun. 16, 2004
(86) PCT No.: PCT/GB04/02595

§ 371 Date: Dec. 16, 2005

(30) Foreign Application Priority Data
Jun. 18, 2003 (GB) 0314131.4

(51) Int. Cl. A61K 31/192; C07C 317/22

(52) U.S. Cl. 514/570; 562/429

(57) ABSTRACT

A potassium salt or a sodium salt of (−)-2-{[2-(4-hydroxyphenyl)ethyl]thio}-3-[4-(2-{4-[(methylsulfonyl)oxy]phenoxy}ethyl)phenyl]propanoic acid, processes for their preparation, their use in treating clinical conditions including lipid disorders (dyslipidemias) whether or not associated with insulin resistance and other manifestations of the metabolic syndrome, and pharmaceutical compositions containing them.

### FIELD OF THE INVENTION

The present invention relates to certain novel salts of (-)-2-{[2-(4-hydroxyphenyl)ethyl]thio}-3-[4-(2-f{4[(methylsulfonyl)oxy]phenoxy}ethyl)phenyl]-propanoic acid, particularly a potassium and a sodium salt thereof, to processes for preparing such compounds, to their the utility in treating clinical conditions including lipid disorders (dyslipidemias) whether or not associated with insulin resistance and other manifestations of the metabolic syndrome, to methods for their therapeutic use and to pharmaceutical compositions containing them

### BACKGROUND OF THE INVENTION

The metabolic syndrome including type 2 diabetes mellitus, refers to a cluster of manifestations including insulin resistance with accompanying hyperinsulinaemia, possibly type 2 diabetes mellitus, arterial hypertension, central (visceral) obesity, dyslipidaemia observed as deranged lipoprotein levels typically characterised by elevated VIDL (very low density lipoproteins), small dense LIDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in patients with the metabolic syndrome and hus to correct the dyslipidaemia which is considered to cause the accelerated progress of atherosclerosis. However, currently this is not a universally accepted diagnosis with well-defmed phanmcotherapeutic indications.

Co-pending PCI application No. PCT/GB02/05743 discloses compounds of formula A 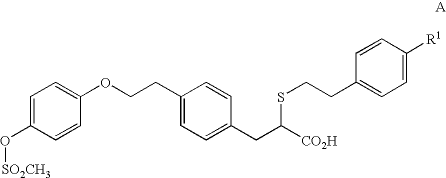
wherein R¹ represents chloro, fluoro or hydroxy as well as optical isomers and racemates thereof as well as pharmaceutically acceptable salts, prodrugs, solvates and crystallie forms thereof which are selective PPARα modulators (for a review of the PPARs (peroxisome proliferator-activated receptors ) see T. M. Willson et al J Med Chem 2000, Vol 43, 527). These compounds are effective in treating conditions associated with insulin resistance. Specific pharmaceutically-acceptable salts of compounds of the formula A are not disclosed in PCT/GB02/05743. Further, no information is provided in relation to how crystalline forms of compounds of the formula A, and particularly salts thereof, may be prepared. The (−) enantiomer of the compound in which R¹ represents hydroxy is prepared as the free acid in this application. However, this compound is a thick oil with a syruplike consistency and is thus not suitable for use in pharmaceutical formulations since it is not mobile. Therefore there exists a need for a solid derivative of this compound which has physical and chemical properties suitable for use in pharmaceutical fonn tons. Many salts were tried but most of these either could not be formed in the solid state or were amorphous with a low glass transition temperature. Salts with suitable propertes for pharmaceutical formulation have now been found.

In the formulation of drug compositions, it is important for the drug substance to be in a form in which it can be conveniently handled and processed. This is of importance, not only from the point of view of obtamin a commercially-viable manufacturing process, but also from the point of view of subsequent manufacture of pharmaceutical formulations comprising the active compound

Further, in the manufacture of drug compositions, it is important that a reliable, reproducible and constant plasma concentration profile of drug is provided following administration to a patient.

Chemical stability, solid state stability, and “shelf life” of the active ingredients are also very important factors. The drag substance, and compositions containing it, should preferably be capable of being effectively stored over appreciable periods of time, without exhibiting a significant change in the active component's physico-chemical characteristics (e.g. its chemical composition, density, hygroscopicity and solubilhy).

Moreover, it is also important to be able to providethe drugin a form which is as chemically pure as possible.

The skilled person will appreciate that, typically, if a drug can be readily obtained in a stable form, such as a stable crystalline form, advantages may be provided, in terms of ease of handling, ease of preparation of suitable pharmaceutical formulations, and a more reliable solubility profile.

### DESCRIPTION OF THE INVENTION

The present invention provides a potassium salt or a sodium salt of (−)-2-{([2-(4-hydroxyphenyl)ethyl]thio}-3-[4-(2-{4[(methylsulfonyl)oxy]phenoxy}ethyl)phenyl]-propanoic acid

According to a further aspect of the invention there is provided a compound of the invention in substantially crystalline form.

It is possible to produce compounds of the invention in forms which are greater than 80% crystalline, by “substantially crystalline” but also included are forms greater than 20%, preferably greater than 30%, and more preferably greater than 40% (e.g. greater than any of 50, 60, 70, 80 or 90%) crystallme.

According to a ihther aspect of the invention there is also provided a compound of the invention in partially crystalline form By “partially crystalline” we include 5% or between 5% and 20% crystalline.

The degree (%) of crystalnnity may be determined by the skilled person using X-ray powder diffraction (XRPD). Other techniques, such as solid state NMR, Fr-IR, Raman spectroscopy, differential scaiin g calorimetry (D)SC) and microcalorimetry, may also be used.

Compounds of the invention, and particularly crystalline compunds of the invention, may have inproved stability when compared to compounds disclosed in PCT/GB02/05743.

The term “stability” as defined herein includes chemical stability and solid state stability.

By “chemical stability”, we include that it may be possible to store compounds of the invention in an isolated form, or in the form of a formulation in which it is provided in admixture with phanmaceutically acceptable carriers, diluents or adjuvants (e.g. in an oral dosage form, such as a tablet, capsule. etc.), under normal storage conditions, with an insignificant degree of chemical degradation or decomposition.

By “solid state stability”, we include that it may be possible to store compounds of the invention in an isolated solid form or in the form of a solid formulation in which it is provided in admixture with pharmaceutically acceptable carriers, diluents or adjuvants (e.g. in an oral dosage form, such as a tablet, capsule etc.), under normal storage conditions, with an insignificant degree of solid state transformation (e.g. crystaUisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, such as 15 to 30° C.), pressures of between 0.1 and 2 bars (preferably at atmospheric pressure), relative humidities of between 5 and 95% (preferably 10 to 60%), and/or exposure to 460 lux of UV/visible light, for prolonged periods (le. greater than or equal to six months). Under such conditions, compounds of the invention may be found to be less than 15%, more preferably less than 10%, and especially less than 5%, chemically degraded/decomposed, or solid state transformed, as appropriate. The killed person will ap ate tt the above mentioned upper and lower liits for temperature, pressure and relative humidity represent extremes of normal storage conditions, and that certain combinations of these extr ns will not be experienced during normal storage (e.g. a temperature of 50° C. and a pressure of 0.1 bar).

It may be possible to crystallise salts of conipomds of the present invention with or without the presence of a solvent system (e.g. crystallisation may be from a melt, under supercritical conditions, or achieved by sublimation). However, we prefer that crystallisation occurs from an appropriate solvent system.

According to a fiurher aspect of the invention, there is provided a process for the preparation of a crystalline compound of the invention which comprises crystallising a compound of the invention from an appropriate solvent system Suitable solvents include ethanol and toluene and mixtures thereof.

Crystalisation ahzes and crystallisation times depend upon the salt that is to be crystallised, the concentration of that salt in solution, and the solvent system which is used.

Crystallisation may also be initiated and/or effected by way of standard techniques, for example with or without seeding with crystals of the appropriate crystalline compound of the invention.

Different crystallie forms of the compounds of the invention may be readily characterised using X-ray powder diffraction (XRPD) methods, for example as described hereinafter.

In order to ensure that a particular crystaline form is prepared in the absence of other crystalline forms, crystallisations are preferably carred out by seeding with nuclei and/or seed crystals of the desired crystalline form in substantially complete absence of nuclei and/or seed crystals of other crystalline forms. Seed crystals of appropriate compound may be prepared, for example, by way of slow evaporation of solvent from a portion of solution of appropriate salt.

Compounds of the invention may be isolated using techniques which are well known to those skilled in the art, for example decanting, filtering or centrifuging.

Compounds may be dried using standard techniques.

Further purification of compounds of the invention may be effected using techniques, which are well known to those skilled in the art. For example impurities may be removed by way of recrystallisation from an appropriate solvent system Suitable temperatures and times for the recrystallisation depend upon the concentration of the salt in solution, and upon the solvent system which is used.

When compounds of the invention are crystallised, or recrystallised, as described herein, the resultant salt may be in a form which has improved chemical and/or solid state stability, as mentioned hereinbefore.

Compounds of the invention have the advantage that they may be more efficacious, be less toxic, be longer acting, have a broader range of activity, be more potent, produce fewer side effects, be more easily absorbed, and/or have a better pharmacokinetic profile (e.g. higher oral bioavailability and/or lower clearance), tha, aundor have other usefuil pharmacological, physical, or chemical, properties over, compounds known in the prior art. Compounds of the invention may have the further advantage that they may be administered less frequently than compounds known in the prior art.

Compounds of the invention may also have the advantage that they are in a form which provides for improved ease of handling. Further, compounds of the invention have the advantage that they may be produced m forms which may have improved chemical and/or solid state stability (including e.g. due to lower hygroscopicity). Thus, such compounds of the invention may be stable when stored over prolonged periods.

Compounds of the invention may also have the advantage that they may be crystallised in good yields, in a high-purity, rapidly, conveniently, and at a low cost.

The compounds of the present invention have activity as medicaments, in particular the compounds are selective agonists of PPARα that is, their EC₅₀ for PPARα is at least ten times lower than their respective EC₅₀ for PPARγ wherein the EC₅₀s are measured and calculated as descnbed in the assays later in this docment. The compounds are potent and selective.

Specific compounds of the invention are:

(−)-2-{[2-(4-hydroxyphenyl)ethyl]thio}-3-[4-(2-{4-[((methylsulfnyl)oxy]-phenoxy}ethyl)phenyl]propanoic acid potassium salt and
(−)-2-{[2-(4-hydroxyphenyl)ethyl]thio}-3-[4-(2-{4-[(methylsuifonyl)oxy]-phenoxy}ethyl)phenyl]propanoic acid sodium salt.

These salts have the advantage that they are crystalline and have good flow characteristics. These salts are sutable for pharmaceutical foo atioll

It will be understood by those skilled in the art that whe (−) occurs in this specification that the acid has a negative rotation when masured using the conditions and concentration described in the experimental section. It should be understood that the salts of the present invention may have (+) rotation provided that the absolute configuration of the salt is the same as the configuration of the (−)-parent acid.

It will also be understood that the compounds of the prsent invention may exist in solvated, for example hydrated or solvated with ethanol, as well as unsolvated forms. It is to be understood that the present invention encompasses all such solvated and unsolvated forms.

In another aspect the invention provides the following embodiments.

A potassium salt of (−)-2-{[2-(4-hydroxyphenyl)ethyl]thio}-3-[4-(2-{4-[(methylsulfonyl)oxy]phenoxy)ethyl)phenyl]propanoic acid char rised by an X-ray powder diffraction pattern characterised by peaks with d-values at 9.3, 5.8, 4.65 and 4.53 Å

A potassinm salt of (−)-2-{[2-(4-hydroxyphenyl)ethyl]thio}-3-[4-(2-{4-[(methylsulfonyl)oxy]phenoxy}ethyl)phenyl]propanoic acid having the XRPD pattern substantially as disclosed in figure A.

A sodium salt of (−)-2-{[2-(4-hydroxyphenyl)ethyl]thio}-3-[4-(2-{4-[(methylsulfonyl)oxy]phenoxy}ethyl)phenyl]propanoic acid chctrised by an X-ray powder diffraction pattern characterised by peaks with d-values at 12.8, 8.2, 4.16 and 4.08 Å

A sodium salt of (−)-2-{[2-(4-hydroxyphenyl)ethyl]thio}-3-[4-(2-{4-[(methylsulfonyl)oxy]phenoxy}ethyl)phenyl]propanoic acid havng the XPD pattern substantially as disclosed in figure B.

Methods of Preparation

The compounds of the invention may be prepared as ot d below. However, the invention is not limited to these methods.

Compounds of the invention may be prepared by reacting (−)-2-([2-(4-hydroxyphenyl)-ethyl]thio)-3-[4-(2-(4-[(methylsulfonyl)oxy]phenoxy}ethyl)phenyl]-propanoic acid with either a potassium containing base, for example potassium hydroxide, or a sodium containing base, for example sodium hydroxide, in an inert solvent at a temperature in the range of 0-100° C. and isolating the solid salt. The salt may be isolated by cooling the reaction solution and optionally seeding the solution with the desired product and/or concentrating the solution. Optionally the product may be isolated by adding an antisolvent to a solution of the product in an inert solvent. The solid may be collected by methods known to those skilled-in the art for example filtration or centrifigation.

In another aspect the present invention provides the compound obtainable by reacting (−)-2-{[2-(4-hydroxyphenyl)ethyl]thio}-3-[4-(2-{[(methylsulfonyl)oxy]phenoxy}-ethyl)phenyl]propanoic acid and potassium hydroxide, in ethanol Particularly an equivalent of potassium hydroxide is used.

In another aspect the present invention provides the compound obtainable by reacting (−)-2-{[2-(4-hydroxyphenyl)ethyl]thio}-3-[4-(2-{-[(methylsulfonyl)oxy]phenoxy}-ethyl)phenyl]propanoic acid and sodium methoxide, in ethanol followed by addition of toluene. Particularly an equivalent of sodium methoxide is used.

The expression “inert solvent” refers to a solvent that does not react with the starting materials, reagents, intermediates or products in a manner which adversely affects the yield of the desired product.

Pharmaceutical Preparations

The compounds of the invention will nomally be administered via the oral, parenteral intravenous, mtramuscular, subcutaneous or in other injectable ways, buccal, rectal, vaginal, transdermal and/or nasal route and/or via inhalation, in the form of pharmaceutical preparations in a pharmaceutically acceptable dosage form Depending upon the disorder and patient to be treated and the route of administration, the compositions may be administered at varying doses.

Suitable daily doses of the compound of the invention in therapeutical treatment of humans are about 0.0001-100 mg/kg body weight, preferably 0.001-10 ng/kg body weight

Oral formulations are preferred particularly tablets or capsules which may be formulated by methods known to those skilled in (be art to provide doses of the active compound in the range of 0.5 mg to 500 mg for example 1 mg, 3 mg, 5 mg, 10 mg, 25 mg, 50 mg, 100 mg and 250 mg.

According to a flier aspect of the invention there is thus provided a pharmaceutical formuilation including the compound of the invention in admixture with pharmaceutically acceptable adjuvants, diluents and/or carriers.

Pharmacological Properties

The compounds of the invention is useful for the prophylaxis and/or treatment of clinical conditions associated with inherent or induced reduced sensitivity to insulin (insulin resistance) and associated metabolic disorders (also known as metabolic syndrome). These clinical conditions will include, but will not be limited to, general obesity; abdominal obesity, arterial hypertension, hyperinsulinaenia, hyperglyeaemia, type 2 diabetes and the dyslipidaemia characteristically appearig with insulin resistance. This dyslipidaemia, also kmown as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoal particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manitations of the metabolic syndrome.

Treatment with the present compoundss is expected to lower the cardiovascular morbidity and mortality associated with atherosclerosis due to their antidyslipidaemic as well as iam tory properties. The cardiovascular disease conditions include macro-angiopathies of various internal organs causing myocardial infarction, congestive heart failure, cerebrovascular disease and peripheral arterial insufficiency of the lower extremities. Because of its insulin sensitizing effect the compound is also expected to prevent or delay the developinent of type 2 diabetes from the metabolic syndrome and diabetes of pregnancy. Therefore the developinent of long-term complications associated with chronic hyperglycaemia in diabetes mellitus such as the micro-angiopathies causing renal disease, retinal damage and peripheral vascular disease of the lower linmbs are expected to be delayed.

Furthermore the compound may be useful in treatment of various conditions outside the cardiovascular system whether or not associated with insuin resistance, like polycystic ovarian syndrome, obesity, cancer and states of infl atory disease including neurodegenerative disorders such as mild cognitive impalnt, Alheimer's disease, Parkinson's disease and multiple sclerosis.

The compounds of the present invention are expected to be useful in controlling glucose levels in patients suffering from type 2 diabetes.

The present invention provides a method of treating or preventing dyslipidemias, the insulin resistance syndrome and/or metabolic disorders (as defined above) comprising the administration of a compound of the present invention to a mammal (particularly a human) in need thereof.

The present invention provides a method of treating or preventing type 2 diabetes comprising the administration of an effective amount of a compound of the present invention to a mammal (particularly a human) in need thereof.

In a further aspect the present invention provides the use of a compound of the present invention as a medicament.

In a further aspect the present invention provides the use of a compound of the present invention in the manufacture of a medicament for the treatment of insulin resistance and/or metabolic disorders.

Combination Therapy

The compounds of the invention may be combined with another therapeutic agent that is useful in the treatment of disorders associated with the developinent and progress of atherosclerosis such as hypertension, hyperlipidaemias, dyslipidaemias, diabetes and obesity. The compound of the invention may be combined with another therapeutic agent that decreases the ratio of LDL:HDL or an agent that causes a decrease in circulating levels of LDL-cholesterol. In patients with diabetes mellitus the compound of the invention may also be combined with therapeutic agents used to treat complications related to micro-angiopathies.

A compound of the invention may be used alongside other therapies for the treatment of metabolic syndrome or type 2 diabetes and its associated complications, these include biguanide drugs, for example metformin, phenformiin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol An example of a prandial glucose regulator is repaglinide or nateglinide.

In another aspect of the invention, the compound of formula I, or a pharmaceutically acceptable salt thereof, may be administered in association with a PPAR modulating agent. PPAR modulating agents include but are not limited to a PPAR alpha and/or ganma and/or delta agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 04/000790, WO 04/000295, WO 04/000294, WO 03/051822, WO 03/051821, WO 02/096863, WO 03/051826, WO 02/085844, WO 01/040172, J Med Chem, 1996, 39, 665, Expert Opinion on Therapeutic Patents, 10 (5), 623-634 (in p lar the compounds- descrbed in the patent applications listed on page 634) and 3 Med Chem, 2000, 43, 527 which are all incorporated herein by reference. Particularly a PPAR alpha and/or gamma a&d/or delta agonist refers to muraglitazar (BMS 298585), rivoglitazone (CS-011), retoglitazone (MCC-555), balaglitazone (DRF-2593, NN-2344), clofibrate, fenofibrate, bezafibrate, gezil, ciprofibrate, pioglitazone, rosiglitazone, AVE-0847, AVE-8134, CLX-0921, DRF-10945, DRF4832, LY-518674, LY-818, LY-929, 641597, GW-590735, GW-677954, GW-501516, MBX-102, ONO-5129, KRP-101, R-483 (BM131258), TAK-559 or TAK-654. Particularly a PPAR alpha and/or gamma and/or delta agonist refers to tesaglitazar ((S)-2-ethoxy-3-[4-(2-{4-methanesulphonyl-oxyphenyl}ethoxy)phenyl]propanoic acid) and pharmaceutically acceptable salts thereof

In addition a compound of the invention may be used in conjunction with a sulfonylurea for example: glimepiride, glibenclamide (glyburide), gliclazide, glipizide, gliquidone, chloropropade, tolbutamide, acetohexaride, glycopyramide, carbutamide, glhbonuride, glisoxepid, glybuthiazole, glibuzole, glyhexamide, glymidine, glypinamide, phenbutamide, tolcylamide and tolazamide. Preferably the sulfonylurea is glimpiide or glibenclamide (glyburide). More preferably the sulfonylurea is glipiride. The present invention includes administration of a compound of the present invention in conjunction with one, two or more existing therapies described in this combination section The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination. The present invention also includes a compound of the present invention in conflation with a cholesterol-lowering agent. The cholesterol-lowering agents referred to in this application include but are not limited to inhibitors of HMG-CoA reductase (3-hydroxy-3-medthylglutaryl coenzyme A reductase). Suitably the HMG-CoA reductase inhibitor is a statin selected from the group consisting of atorvastatin, bervastatin, cerivastatin, dalvastatin, fluvastatin, itavastatin, lovastatin, mevastatin, nicostatin, nivastatin, pravastatin and simvastatin, or a pharmaceutically acceptable salt, especially sodium or calciU3A or a solvate thereof, or a solvate of such a salt. A particular statin is atorvastatin, or a pharmaceutically acceptable salt, solvate, solvate of such a salt or a prodrug thereof A more particular statin is atorvastatin calcium salt. A particularly preferred statin is, however, a compound with the chemical name (E)-7-[4-(4-fluorophenyl)-6-isopropyl-2-[methyl(methylonyl)-amino]-pyrimidin-5-yl](3R,5S)-3,5-dihydroxyhept-6-enoic acid, [also known as (E)-7-[4-(4-fluorophenyl)-6-isopropyl-2-[N-methyl-N-(methylsulfonyl)-amino]pyrimidin-5-yl](3R,5S)-3,5-dihydroxyhept-6-enoic acid] or a pharmaceutically acceptable salt or solvate thereof, or a solvate of such a salt. The compound (E)-7-[4-(4fluorophenyl)-6isopropyl-2-[methyl-(methylsulfonyl)-amino]-pyrimidin-5-yl](3R,5S)-3,5-dihydroxyhept-6-enoic acid, and its calcium and sodium salts are disclosed in European Patent Application, Publication No. EP-A-0521471, and in Bioorganic and Medicinal Chemistry, (1997), 5(2), 437-444. This latter statin is now known under its generic name rosuvastatin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with a bile acid sequestering agent, for example colestipol or cholestyramine or cholestagel.

The present invention also includes a compoimd of the present invention in comfoination with an inhibitor of the ileal bile acid transport system (IBAT inhibitor).

Suitable compounds possessing IBAT inlnhbitory activity have been described, see for instance the compounds described in WO 93116055, WO 94/18183, WO 94/18184, WO 96/05188, WO 96/08484, WO 96/16051, WO 97/33882, WO 98/07449, WO 98/03818, WO 98/38182, WO 99/32478, WO 99/35135, WO 98/40375, WO 99/35153, WO 99/64409, WO 99/64410, WO 00/01687, WO 00147568, WO 00/61568, WO 00/62810, WO 01/68906, DE 19825804, WO 00/38725, WO 00/38726, WO 00/38727, WO 00/38728, WO 00/38729, WO 01/68906, WO 01/66533, WO 02/32428, WO 02/50051, EP 864 582, EP489423, EP549967, EP573848, EP624593, EP624594, EP624595 and BP624596 and the contents of these patent applications are incorporated herein by reference.

Further suitable compounds possessmg IBAT inhibitory activity have been described in WO 94/24087, WO 98/56757, WO 00/20392, WO 00120393, WO 00/20410, WO 00/20437, WO 0134570, WO 00/35889, WO 01/68637, WO 02/08211, WO 03/020710, WO 03/022825, WO 03/022830, WO 03/022286, WO 03/091232, WO 03/106482, JP 10072371, U.S. Pat. No. 5,070,103, EP 251 315, EP 417 725, BP 869 121, EP 1 070 703 and EP 597 107 and the contents of these patent applications are incorporated herein by reference.

Particular classes of IBAT inbitors suitable for use in the present invention are benzothiepines, and the compounds described in the claims, particularly claim 1, of WO 00/01687, WO 96/08484 and WO 97/33882 are incorporated herein by reference. Other suitable classes of MBAT inhibitors are the 1,2-benzothiazepines, 1,4-benzothiazepines and 1,5-benzothiazepines. A further suitable class of IBAT inhibitors is the 1,2,5-benzot epines.

One particular suitable conpound possessing IBAT izihibtory activity is (3R,5R)-3-butyl-3-ethyl-1,1-dioxido-5-phenyl-2,3,4,5-tetrahydro-1,4-benzothiawepin-8-yl P-D-glucopyranosiduronic acid (EP 864 582). Other suitable BAT inbibitors include one of: 1,1-dioxo-3,3-dibutyl-5-phenyl-7-methylthio-8-(N-{(R)-1′-phenyl-1′-[N′-(carboxymethyl) carbamoyl}methyl]carbamoylnethoxy)-2,3,4,5-tetrahydro-1,5-benzothiazepine; 1,1-dioxo-3,3-dibutyl-5-phenyl-7-methyithio-8-(N-{(R)-1-[N′-(carboxymethyl)carbamoyl]-4-hydroxybenzyl}carbamoylmetoxy)-2,3,4,5-tetrahydro-1,5-benzothizepine; 1,1-dioxo-3,3-dibutyl-5-phenyl-7-methylthio-8-(N-{(R)-1′-phenyl-1-[N-(2-subphoethyl)carbamoyl]methyl}carbamoylmethoxy)-2,3,4,5-tetrahydro-1,5-benzothiazepine; 1,1-dioxo-3-butyl-3-ethyl-5-phenyl-7-methylthio-8-(N-{(R)-1′-phenyl-1′-[N′-(2-sulphoethyl)carbamoyl]methyl}carbamoyl]methoxy)-2,3,4,5-tetrahydro-1,5-benzothiazepine; 1,1-dioxo-3,3-dibutyl-5-phenyl-7-methylthio-8-(N-{(R)-α-[N′-(2-sulphoethyl}carbamoyl]-4-hydroxybenzyl}carbamoylmethoxy)-2,3,4,5-tetudiydro-1,5-benzothiazepine; 1,1-dioxo-3-butyl-3-ethyl-5-phenyl-7-methylthio-8-(N-{(R)-α-[N′-(2-sulphoethyl}carbamoyl]-4-hydroxybenzyl)carbamoylmethoxy)-2,3,4,5-tetrahydro-1,5-benzothiazepine; 1,1-dioxo-3-butyl-3-ethyl-5-plenyl-7-methylthio-8-(N-(R)-α-[N′-(2-carboxyethyl)carbamoyl]benzyl)carbamoybmehoxy)-2,3,4,5-tetrahydro-1,5-benzothiazepine; 1,1-dioxo-3,3-dibutyl-5-phenyl-7-metylthio-8-(N-{(R)-α-[M-(2-carboxyethylicarbamoyl]-4-hydroxybenzyl)carbainoylmethoxy)-2,3,4,5-tetrahydro-1,5-benzothiazepine; 1,1-dioxo-3-butyl-3-ethyl-5-phenyl-7-methylthio-8-(N-{(R)-α-[N′-(5-arboxypentyl) carbamoyl]benzyl}carbamoylmethoxy)-2,3,4,5-tetahydro-1,5-benzothiazepine; 1,1-dioxo-3,3-dibutyl-5-phenyl-7-methylthio-8-(N-(R)-α-[N′-(2-carboxyethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{a-[N′-(2-sulphoethyl)carbamoyll-2-fluorobenzyl)carbamoylmethoxy)-2,3,4,5-tahydro-1,5-benzothiazepine; 1,1-dioxo-3-butyl-3-ethyl-5-phenyl-7-methylthio-8-(N-(R)-α-(N′-[N)-(2-hydroxy-1-carboxyethyl)carbamoyl]benzyl}carbamoyl]methoxy)-2,3,4,5-tetrahydro-1,5-benzothiazepine; 1,1-dioxo-3,3-dibutyl-5-phenyl-7-methyltlio-8-(N-{(R)-[N′-)-(2-hydroxy-1-carboxyethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl]carbamoyl)benzyiacarbamoybmethoxy}-2,3,4,5-tetrahydro-1,5-bezothiazepine; 1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{α-[N′-((ethoxy)(methyl)phosphoryl-methyl}carbamoyl]benzyl)carbamoylmethoxy)-2,3,4,5-tetrahydro-1,5-benzothiazepine; 1,1-dioxo-3-butyl-3-ethyl-5-phenyl-7-methylthio-8-{N-[(R)-α-({2-[(hydroxy)(methyl)phosphoryl)ethyl}carbamoyl)benzyl]carbamoylmethoxy}-2,3,4,5-tetrahydro-1,S-benzothiazepine; 1,1-dioxo-3,3-dibutyl-5-phenyl-7-methylthio-8-(N-{(R)-α-[N-(2-methylthio-1-carboxyethyl)carbaoyl]benzyl}carbamoyl]methoxy)-2,3,4,5-tetrahydro-1,5-benzothiazepine; 1,1-dioxo-3,3-dibutyl-5-phenyl-7-methylthio-8-{N-[(R)-α-N′-{2-[(methyl)(ethyl) phosphoryl]ethyl}carbamoyl)-4-hydroxybenzyl]carbyhmethoxy}-2,3,4,5-tetrahydro-1,5-benzothiazepine; 1,1-dioxo-3,3-dibutyl-5-phenyl-7-methylthio-8-{N-[(R)-α-N′-{2-[(raethyl)(hydroxy) phosphoryl]ethyl}carbamoyl)-4-hydroxybenzyl]carbamoyhnethoxy}-2,3,4,5-tetrahydro 1,5-benzothiazepine; 1,1-dioxo-3,3-dibutyl-5-phenyl-7-methylthio-8-(N-{(R)-α-[(R)-1-(2-mthylsulphinyl-1-carboxyethyl)carbamoyl]benzyl)carbamoyhmethoxy)-2,3,4,5-ethydro-1,5-benzothiazepine; 1,1-dioxo-3,3-dibutyl-5-phenyl-7-methoxy-8-[N-{(R)-α-[N′-(2-sulphoethyl)carbamoyl]-4-hydroxybenzyl)carbamoylmethoxy]-2,3,4,5-tetrahydro-1,5-benzothiazepine; 1,1-dioxo-3,3-dibutyl-5-phenyl-7-methylthio-8-(N-{(R)-α-[N-((R)-1-carboxy-2-methylthio-ethyl)carbamoyl]-4-hydroxybenzyl}carbamoybmethoxy)-2,3,4,5-tetrahydro-1,2,5-benzothiadiazepine; 1,1-dioxo-3,3-dibutyl-5-phenyl-7-methylthio-8-(N-{(R)-α-[N-((S)-1-carboxy-2-(R)-hydroxypropyl)carbamoyl]-4-hydroxybenzyl)carbamoylmebtoxy)-2,3,4,5-tetrahydro-1,2,5-benzothiadiazepine; 1,1-dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-etrahydro-1,2,5-benzotfmiadiazeine; 1,1-dioxo-3,3-dibutyl-5-phenyl-7-methylthio-8-(N-{(R)-α-[N-(S)-1carboxypropyl) carbamoyl]benzyl}carbanoybnethoxy)-2,3,4,5-tetrahydro-1,2,5-benzothiadiazepine; 1,1-dioxo-3,3 dibutyl-5-phenyl-7-methylthio-8-(N-{(R)-α-[N-((S)-1-carboxyethyl) carbamoyl]benzyl}carbamoyhmethoxy)-2,3,4,5-etrahydro-1,2,5-benzothiadiazepne; 1,1-dioxo-3,3-dibiutyl-5-phenyl-7-=thylthio-8-(N-{(R)-α-[N-((S)-1-carboxy-2-(R)-hydroxypropyl)carbamoyl]benzyl)carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-2-sulphoethyl)carbamoyl]4-hydroxybenzyl}carbamoybmethoxy)-2,3,4,5-tetrahydro-1,2,5-benzothiadiazepine; 1,1-dioxo-3,3-dibutyl-5-phenyl-7-methylthio-8-(N-{(R)-α-[N-(S)-1-carboxyethyl)carbamoyl]hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(R)-1-carboxy-2-methylthioethyb) carbamyllbenyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(S)-1-[N-((S)2-hydroxy-1-carboxyethyl)carbamoyl]propyl}earbamoyl]benzyllcalbamoyhliethoxy)-2,3,4,5tetrahydro-1,2,5-benzothiaiazepine; 1,1-dioxo-3,3-dibutyl-5-phenyl-7-methylthio-8-(N-{(R)-α-[N-(S)-1-carboxy-2-methylpropyl)carbamoyllbenzyl}carbamoylmethoxy)-2,3,4,5-teydro-1,2,5-benzothiadiazepine; 1,1-Dioxo-3,3-dibutyl-5-phenyl-7-methylthio-8-(N-{(R)-α-[N-((S)-1-carboxypropyl) carbamoyl]4-hydroxybenzyl}carbamaylmethoxy)-2,3,4,5-tetrahydro-1,2,5-benzothiadiazepine; 1,1-Dioxo-3,3-dibutyl-5-phenyl-7-methylthio-8-[N-((R/S)-o {N-[1-(R)-2-(S)-1-hydroxy-1-5 (3,4dihydroxyphenyl)prop-2-yl]carbamoyl)hydroxybenzyl)carbamoylmethoxy]-2,3,4,5-tetrahydro-1,2,5-benzothiadiazepine; 1,1-Dioxo-3,3-dibutyl-5-phenyl-7-methylthio-8-(N-{(R)-ct-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine; and 1,1-Dioxo-3,3-dibutyl-5-phenyl-7-methylthio-8-(N-I(R)-α-[N-(2-(S)-3-(R )-5-(R)-2,3,4,5,6pentahydroxyhexyI)carbamoyllbenzyl}carbamoylmethoxy)-2,3,4,5-tetrahydro-1,2,5-benzothiadiazepine; or a pharmaceutically acceptable salt, solvate, solvate of such a salt or a prodnig thereof

According to an additional further aspect of the present invention there is provided a combination treament comprising the administration of an effective amount of a compound of the present invention optionally together with a pharmaceutically acceptable diluent or carrier, with the simultaneous, sequential or separate administration one or more of the following agents selected from:
a CETP (cholesteryl ester transfer protein) inibitor, for example those referenced and desc1ibed in WO 00/38725 page 7 line 22—page 10, line 17 which are incorporated herein by reference;
a cholesterol absorption antagonist for example azetidinones such as SCH 58235 and those described in U.S. Pat. No. 5,767,115 which are incorporated herein by reference;
a MTP (microsomal transfer protein) inhibitor for example those described in Science, 282, 751-54, 1998 which are incorporated herein by reference;
a nicotinic acid derivative, including slow release and combination products, for example, nicotinic acid (niacin), acipimox and niceritrol;
a phytosterol compound for example spools; probucol;
an omega-3 fatty acid for example Omacor™;
an anti-obesity compound for example orlistat (EP 129,748) and sibutramine (GB 2,184,122 and U.S. Pat. No. 4,929,629);
an antihypertensive compound for example an angiotensin converting enzyme (ACE) ihi1bitor, an angiotensin II receptor antagonist, an andrenergic blocker, an alpha andrenergic blocker, a beta andrenergic blocker for exancple metoprolol, a mixed alpha/beta andrenergic blocker, an andrenergic stimulant, calcium channel blocker, an AT-1 blocker, a saluretic, a diuretic or a vasodilator;
a CB1 antagonist or inverse agonist for example as described in wooio700 and EP 65635
asprin;
a Melanin concentrating hormone (MCH) antagonist;
a PDK inhbitor, or
modulators of nuclear receptors for example FXR, RXR, and RORalpha;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Particular ACE inhibitors or pharmaceutically acceptable salts, solvates, solvate of such salts or a prodrugs thereof, including active metabolites, which can be used in combinion with a compound of the invention include but are not limited to, the following compounds: alacepril, alatriopril, altioprfl calcim, ancoveni, benazepril, benazepril hydrochloride, benazeprilat, benzoylcaptopril, captopril, captopril-cysteine, captopril-glutathione, ceranapril, ceranopril, ceronapil, cflazapril cilazaprilat, delapril, delapil-diacid, enalaprll, enalaprilat, enapril, epicaptopril, foroxyimine, fosfenopril fosenopriL fosenopril sodium, fosinopril, fosinopril sodium, fosinoprilat, fosinopiic acid, glycopril, hemorphin-4, idrapril, imidapril, indolapril, indolaprilat, libenzapri, lisinopril, lycium A, lyciuimin B, mixan, moexipril moexiprilat, moveltipril, maracein A, muracein B, muracein C, pentopril, perindopril, perindoprilat; pivalopril, pivopril qu quinapril hydrochloride, qumaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril zabicipril, zabiciprilat, zofenopril and zofenoprilat. Preferred ACIB inhibitors for use in the present invention are ramipril, raat, lisinopril, enalapril and enalaprilal more preferred ACE inhibitors for uses in the present invention are ramipril and ramipritlat.

Preferred angiotensin II antagonists, pharaceuticauy acceptable salts, solvates, solvate of such salts or a prodrugs thereof for use in combination with a compound of e inve ntion inchlde, but are not limited to, compounds: candesartn, cnesartan cilexetil, losartan, valsartan, irbesartan, tasosaan, telmisartan and eprosartam Particularly preferred angiotensin II antagonists or pharmaceutically acceptable derivatives thereof for use in the present invention are candesartan and candesartan cilexetil.

Therefore in an additional feature of the invention, there is provided a method for for the treatment of type 2 diabetes and its associated complications in a warm-blooded aninal, such as man, in need of such treatment which comprises administering to said animal an effective amount of a compound of the present invention in simultaneous, sequential or separate administration with an effective amount of one the other compounds described in this combination section, or a pharmaceutically acceptable salt, solvate, solvate of such a salt or a prodrug thereof

Therefore in an additional feature of the invention, there is provided a method of treating hyperlipidemic conditions in a warmblooded animal, such as man, in need of such treatment which comprises administering to said animal an effective amount of a compound of the present invention of a compound of the invention in simultaneous, sequential or separate adminison with an effective amount of one the other compounds described in this combination section or a pharmaceutically acceptable salt, solvate, solvate of such a salt or a prodrug thereof.

According to a firtr aspect of the invention there is provided a pharmaceutical composition which comprises a compound of the present invention and one of the other wo compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of the present invention and one of the other compounds described in this combination section or a pharmaceutically acceptable salt, solvate, solvate of such a salt or a prodrug thereof.

According to a fuirther aspect of the present invention there is provided a kit comprising:
a) a compound of the present invention in a first unit dosage form;
b) one of the other compounds described in this combination section or a pharmaceutically acceptable salt, solvate, solvate of such a salt or a prodrug thereof; in a second unit dosage form; and
c) container means for containing said first and second dosage forms.

According to a fierceer aspect of the present invention there is provided a kit comprising:
a) a compound of the present invention together with a pharmaceutically acceptable diluent or carrier, in a first unit dosage form;
b) one of the other compounds described in this combination section or a pharmaceutically acceptable salt, solvate, solvate of such a salt or a prodrug thereof, in a second unit dosage form; and
c) container means for containing said first and second dosage forms.

According to anotier feature of the invention there is provided the use of a compound of the present invention of the present invention and one of the other compounds described in this combination section, or a pharmaceutically acceptable salt, solvate, solvate of such a salt or a prodrug thereof, in the manufacture of a mfdicament for use in the the treatment of metabolic syndrome or type 2 diabetes and its associated complications in a wann blooded animal, such as man.

According to another feature of the invention there is provided the use of a compound of the present invention and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal such as man.

According to a furter aspect of the present invention there is provided a combination treatment comprisig the administration of an effective amount of a compound of the present invention optionally together with a phaceutically acceptable diluent or carrier, with the simultaneous, sequential or separate administration of an effective amount of one of the other compounds described in this combination section, or a pha mally acceptable salt, solvate, solvate of such a salt or a prodrug thereof, optionally together with a pharmaceutically acceptable diluent or carrer to a warm blooded animal, such as man in need of such therapeutic treatment.

Exerimental

¹H NMR and ¹³C NMR mesretents were performed on a Vaian Mercury 300 or Varian UNITY plus 400, 500 or 600 spectrometers, operating at ¹H frequencies of 300, 400, 500 and 600 MHz, rectively, and at ¹³C frequencies of 75, 100, 125 and 150 MHz, respectively. Measurements were made on the delta scale (δ).

Unless otherwise stated, chemical shifts are given in ppm with the solvent as internal standard.

X-ray powder diffraction analysis (XRPD) was performed using variable slits on samples prepared according to standard methods without using any internal standard. Standard methods are described in, for example, Giacovazzo, C. et al (1995), Fundamentals of Crystallography, Oxford University Press; Jenkins, R. and Snyder, R. L. (1996), Introduction to X-Ra)y Powder Diffractometry, John Wiley & Sons,. New York; Bu, C. W. (1948), Chemical Crystallography, Clarendon Press, London; or Kiug, H. P. & Alexander, L. E. (1974), X-ray Diffraction Procedures, John Wiley and Sons, New York. X-ray analyses 10 were performed using Cu-radiation on a Siemens D5000 diffractometer. The X-axis in the figures below is 2-theta and the Y-axis is intensity. Differential scanning calorimetry (DSC) was performed using a Mettler DSC820 or a Mettler DSC820E, according to standard methods, for example those described in Höhne, G. W. H. et al (1996), Differential Scanning Calorimetry, Springer, Berlin Tbermo-gravimetric analysis (TGA) was performed using a Mettler Toledo TGA850 or a Mettler Toledo TG851. A ranm rate of 10° C./min was used. It will be appreciated by the skilled person that crystalline forms of compounds of the invention may be prepared by analogy with processes described herein and/or in accordance with the Examples below, and may show essentially the same XRPD diffraction patterns and/or DSC and/or TGA thermograms as those disclosed herei By “essentially the same” XRPD diffraction patterns and/or DSC and/or TGA thermograms, we include those instances when it is clear from the relevant patterns and/or thermograms (allowing for experimental error) that essentially the same crystalline form has been formed When provided, DSC onset temperatures may vary in the range ±5° C. (e.g. ±2° C.), and XRPD distance values may vary in the range ±2 on the last decimal place. It will be appreciated by the skilled person that XRPD intensities may vary when measured for essentially the same crystalline form for a variety of reasons including, for example, preferred orientation

Abbreviations

| | |
|---|---|
| DMSO | dimethyl sulfoxide |
| EtOAc | ethyl acetate |
| DMF | N,N-dimethylformamide |
| THF | tetrahydrofuran |
| MeCN | acetonitrile |
| MeOH | methanol |
| TFA | trifluoroacetic acid |
| NH₄OAc | ammonium acetate |

NMR Abbreviations

| | |
|---|---|
| t | triplet |
| s | singlet |
| d | doublet |
| q | quartet |
| m | multiplet |
| bs | broad singlet |

XRPD Abbreviations
XRFD X-ray powder diffraction

d-value the spacing between successive parallel hkl planes in a crystal lattice

| Intensity (rel %) | Definition | |
|---|---|---|
| 25-100 | vs | (very strong) |
| 10-25 | s | (strong) |
| 3-10 | m | (medium) |
| 1-3 | w | (weak) |

TGA thermogravimetric analysis
DSC differential scanning calorimetry

### EXAMPLES

Preparation of Acid Starting Material

(i) Methyl2-chloro-3-[4-(2-hydroxyethyl)penyl]prmpanoate

2-(4Aminophenyl)ethanol (11 g, 81 mmol) and 32 ml conc HCl was dissolved in acetone and cooled to 0° C. Sodium nitrite (5.6 g, 81 mmol) in 20 ml water was added dropwise. The temperature was kept under 0° C. After one hour, methyl acrylate (70 g, 808 mmol) and CuI (1.6 g, 8 mmol) were added (<0° C.). The reaction mixture was stirred at room temperature overnight.

The solvent was evaporated and water was added. The water phase was extracted three times with EtOAc, the organic phases were pooled and wasbed with water, dried (MgSO₄) and evaporated under reduced pressure. The crude product was purified by flash chromatography using a 65:35 mixture of EtOAc and heptane as eluent. Putle purification by preparative BPLC (using a gradient of CH₃CN/5%CH3CN-waterphase containing 0.1M NH₄OAc as eluent) gave 9.7 g product (yield 49%) as an oil.
¹HNMR (400 MHz, CDC13) 2.84 (t, 3H), 3.15 (dd, 1H), 3.35 (dd, 1H), 3.75 (s, 3H), 3.84 (t, 3H), 4.43 (t, 1H), 7.17 (d, 4M
   (ii) Methyl 3-(4-12-(benzyloxn)phenoxylethyl]phenyl)-2-chloropropanoate

Triphenylphosphine (2.4 g, 9 mmol) was added to a solution of methyl 2-chloro-3-[4-(2-hydroxyethyl)phenyl]propanoate (2.1 lg, 8.5 mmol) and 4-(benzyloxy)phenol (1.7 g, 8 mmol) in 20 ml toluene under nitrogen atmosphere. The solution was warmed to 55° C. and diisopropyl azodicarboxylate (1.8 g, 9nmo) was added. The reaction mixture was stirred at 55° C. overnight.

The mixture was allowed to cool and the solvent was evaporated under reduced pressure. The crude product was purified by flash chromatography using a 80:20 mixture of heptane and EtOAc as eluent to yield 2.28 g of the desired product (yield 61%) as colourless crystals.
¹HNMR (400 MHz, CDCl₃): 3.05 (t, 2H), 3.16 (dd, 1H), 3.36 (dd, 1H), 3.75 (s, 3H), 4.12 (t, 2H), 4.45 (t, 1H), 5.01 (s, 2H), 6.82 (i, 2H), 6.90 (m, 2H), 7.13-7.27 (m, 4H), 7.29-7.47 (m, 5H).
   (iii) Methyl 2-chloro-3-{4-[2-(4-hydroxyphenoxy)ethyl]phenyl}propanoate

Methyl 3-(4-{²-[⁴-(benzyloxy)phenoxyjethyl)phenyl)-2-chloropropanoate (1.0 g, 2.4 mmol) and dimethyl sulfide (0.9 g, 14 mmol) was dissolved in 60 ml CH₂Cl₂. Boron trifluoride etherate (2.0 g, 14 mmol) was added droppwise to the stirred solutiom The reaction mixture was stirrred for two days at room temperature. Another equivalent (0.4 g, 2.87 mmol) boron trifluoride etherate was added and the stirrig was continued overnight.

Water was added. The phases were separated and the aqueous phase was extracted twice with CH₂Cl₂. The organic phases were pooled, washed (water, brine), dried (Na₂SO₄) and evaporated under reduced pressure. Futher purification by preparative BPLC using a gradient of CH3CN/5% CH₃CN-waterphase contang 0.1 M NH₄OAc gave 0.55 g of the desired product (yield 52%) as an oil.
¹HNMR (400 MHz, CDCl₃ ): 3.04 (t, 2H), 3.16 (dd, 1H), 3.35 (dd, 111), 3.75 (s, 3H), 4.10 (t, 21), 4.40 (t, 1H), 6.75 (m, 4H), 7.12-7.29 (m, 4H).
   (iv) Methyl 2-chloro-3-[4-(2-(4-[(mefthylsulfonyl)oxy]phenoxylethyl)phenyl]propanoate

Methyl 2-chloro-3-{4-[2-(4-hydroxyphenoxy)ethyl]phenyipropanoate (334mpg, 1.0 mmol) and triethylamine (303nmg, 3.0 mmol) was dissolved in 20 ml dichlormethane and cooled to −20° C. umder nitrogen atmosphere. Methaneonyl chloride (1 14 mg, 1.0 mmol) was added dropwise. The mixture was allowed to reach room temperature. After 2 hours dichlormethane was added, the mixture was washed (water, brine), dried (Na2SO₄) aiid evaporated umder reduced pressure to yield 394 mg pure product (yield 96%).
¹HNMk (400Mz, CDCl₃): 3.02-3.11 (m,SH), 3.15 (dd, 1H), 3.35 (dd,1H), 3.74 (s, 3H), 4.14 (t, 2H), 4.44 (t, 1H), 5.29 (s, 2H), 6.88 (d, 2H), 7.14-7.25 (m, 6H).
   (v) Methyl 2-({2-[4-(benzyloxy)phenyl]ethyl}thio)-3-[4-(2-[4-[(methylsulfonyl)oxy]phenoxy}ethyl)phenyl]propanoate

2-[4-(Benzyloxy)phenyl)ethanethiol (334 mg, 1.4 mmol), methyl 2-chloro-3-[4-(2-{4-[(methylsulfonyl)oxy]phenoxy}ethyl)phenyl]propanoate (394 mg, 0.95 mmol) and potassium carbonate (189 mg, 1.4 mmol) were dissolved in 14 ml dry DMF and stirred under nitrogen atmosphere at room temperature ovemight.

The solvent was evaporated under reduced pressure and the residue was dissolved in toluene. The organic phase was washed (water, brine), dried (MgSO₄) and evaporated. Further purification by preparative HPLC usmg a gradient of CH₃CN/5% CH3CN-waterphase containing 0.1 M NH₄OAc gave 477 mg of the desired product (yield 75%).
¹HNMR (400Mz, CDCl₃ ): 2.762.89 (m, 4H), 2.95 (dd, 1H), 3.09 (m,SH), 3.20-(dd, 1H), 3.53 (m, 1H), 3.70 (s, 3H), 4.15 (t, 2H), 5.06 (s, 2H), 6.91 (m, 4H), 7.07-7.24 (m, 8H), 7.31-30 7.48 (m, 5H).
   (vi) Methyl 2-{[2-(4-hydroxyphenyl)ethyl]thio}-3-[4-(2-{4-[(methylsulfonyloxy]phenoxyethyl)phenyl]propanoate

To a solution of methyl 2-({2-[4-(benzyloxy)phenyl)ethyl}thio)-3-[4-(2-{4-[(methylsulfonyl)oxy]phenoxy}ethyl)phenyl]propanoate (477 mg, 0.8 mmol) and 15 ml dichlormethane, dimethyl sulfide (239 mg, 3.8mol) and boron trifluoride etherate (545 mg, 3.8 mmol) were added. After 18 hours of stirrig water was added to the reactior The phases were separated and the aqueous phase was extracted twice with dichlormethane. The organic phases were pooled, dried (MgSO4) and evaporated under reduced pressure. 274 mg of the desired product (yield 67%) was obtained as an oil. ¹HNMR (400 MHz, CDCl₃): 2.70-2.85 (m, 4H), 2.91 (dd, 1H), 3.05 (t, 2H), 3.10 (s, 3H), 3.17 (dd, 1H), 3.49 (nm, 1), 3.68 (s, 31), 4.13 (t, 2H), 6.72 (d, 2H), 6.87 (d, 2M), 6.99 (d, 2H), 7.107.22 (m, 6H)

(vii) 2-{[2-(4-Hydroxyphenyl)ethyl]thio 1-3-[4-(2-[4-(methylsulfonyl)oxy]-phenoxy}ethyl]phenyl]propanoic acid

Methyl 2-{[2-(4-hydroxyphenyl)ethyl]thio )-3-[4-(2-(4-[(methylsulfonyl)oxy]phenoxy}ethyl)phenyl]propanoate (105 mg, 0.2 mmol) was dissolved in 6.5 ml of a 7:1 mixture of TBF and water and cooled on an ice-bath Lithium hydroxide (9.4 mg, 0.4 mmol) was added. Water was added to the reaction mixwure after 24 hours of stirring at room temperature. The THF was evaporated under reduced pressure and the residue was acidified with 1M hydrochloric acid. The water phase was extracted with EtOAc (×3), the organic phases were pooled, washed (water, brine), dried (MgSO4) and evaporated. The crude product was purified using preparative HPLC (eluent: CH3CN/5% CH3CN-waterphase containing 0.1M NH₄OAc) to give 74 mg of the desired product (yield 97%) as an oiL
¹HNMR (400 MHz, CDC1₃): 2.68-2.95 (m, 51), 3.05 (t, 2H), 3.10 (s, 3H), 3.17 (dd, 1H), 3.47 (m, 1H), 4.12 (t, 2H), 6.70 (d, 2H), 6.86 (d, 2H), 6.97 (d, 2H), 7.12-7.21 (m, 6H).
¹³CNMR (100 MHz, CDCl₃): 33.8, 35.1, 35.5, 37.2, 37.3, 48.1, 69.3, 115.6, 115.8, 123.3, 129.3, 129.4, 129.9, 132.3, 136.2, 136.9, 142.8, 154.4, 158.0, 177.2.
   (viii) (−)-2-[2-(4-hydroxyphenyl)ethyl]thio}-3-[4-(2-[4-[(methylsulfonyl)oxy]phenoxylethyl)pheny1propanoic acid

The racemate of 2-(2-(4-hydroxyphenyl)ethy])thio}-3-[4-(2-(4[(=ethylsulfonyl)-5 oxy]phenoxy)ethyl)phenyl]propanoic acid was separated into its enantiomers using chiral chromatography. A Chiralpak AD JDBO1+AS003 (336×100 mm id.) and ethanoilformic acid 100/0.01% was used as mobile phase. The racemate (9 g) was dissolved in ethanol anid injected onto the column. The first eluting peak was collected and UV-detected The product (4.1 g) was obtained with an enantiomeric purity >99%. The optical rotation was found to be [α]²⁰_{D}=33° by dissolving the enantiomer in methanol to give a concentration of 0.64 g/100 ml The optical rotation was measured at 20° C. using Whe sodium line at 589 mim
¹H NMR (500 MHz, CD₃OD): 7.17-7.22 (6H, m), 6.99 (2H, d), 6.94 (2H, d), 6.69 (2H, d), 4.17 (2H, t), 3.46 (1H, t), 3.16 (3H, s), 3.13 (1H, dd), 3.05 (2H, t), 2.69-2.88 (5H, m).

### Examule 1

Potassium salt of (−)-2-{[2-(4-hydroxypenyl)ethyl]thio}-3-[4-(2-[4-[(methylsulfonyl)oxy]phenoxy}ethyl)phenyl]propanoic acid

A solution of (S)-2-{[2-(4-hydroxyphenyl)ethyl]thio}-3-[4-(2-{4-[(methylsulfonyl)oxy]-phenoxy)ethyl)phenyl]propanoic acid (344 g). in ethanol (8.2 L) was neutralieed with addition of ethanolic 4.2% w/w potassium hydroxide solution (1.2 L). The neutralised solution was heated to 50° C. and screened into a crystalliser vessel The solution was reduced in volume to 4.4 L by distillation. Whilst still hot at 50° C., toluene (6.2 L) was added as co-solvent. The batch was allowed to cool to anibient temperature and seeded. urther toluene (4.2 L) was added to the crystallised solutions The slurry was chilled to −5° C. over 60 minutes and held for a further 150 minutes. The product was isolated by filtration and washed with chilled toluene. The reaction afforded title compound as an off white solid in 339 g yield.

The X-axis in the figures below is below is 2-theta and the Y axis is intensity. DSC showed an endotherm with an extrapolated onset temperature in the range of 91-97° C. TGA showed a weight loss in the range of 6.0-7.1% w/w between 25-110° C. DSC analysis repeated on purer sample may give. a higher melting point

Crystals of the potassium salt of (S)-2-[2-(4-hydroxyphenyl)ethyl]thio}-3-[4-(2-{4-[(methylsulfonyl)oxy]-phenoxy}ethyl)phenyl]propanoic acid (obtained by way of the example above and/or by other ways) were analyzed by XRPD and the results are tabulated below and are shown in Figure A.

[image]

Figure A, XRD pattern of potassium salt of (S)-2-{[2-(4-hydroxyphenyl)ethyl]thio}-3-[4-(2-(4-[(methylsulfonyl)oxy]phenoxy}ethyl)phenyl]propanoic acid

| d-value | intensity |
|---|---|
| (Angstrom) | (rel) |
| 17.4 | w |
| 9.3 | vs |
| 7.1 | w |
| 6.8 | m |
| 6.4 | w |
| 5.8 | m |
| 5.1 | m |
| 4.65 | m |
| 4.53 | s |
| 4.29 | m |
| 3.99 | w |
| 3.91 | w |
| 3.61 | w |
| 3.55 | m |
| 3.39 | w |
| 3.12 | m |

### Example 2

### Sodium salt of (−)-2-{[2-(4-hydroxyphenyl)ethyl]thio}-3-[4-(2-{4-[(methylulfonyl)oxyl-phenoxy}ethyl)phenyl]propanoic acid

(−)-2-([2-(4-hydroxyphenyl)ethyl]thio}-3-[4-(2-{4[(methylsulfonyl)oxy]-phenoxy}ethyl)phenyl]propanoic acid (529 mg) was dissolved in ethanol (3.2 D. The solution was heated to 45° C. and sodium methoxide (58 mg, 1.03 eq.) was added. Then, the solution was heated to 60° C. and toluene was added (4 ml). After this, the solution was cooled slowly to 0° C. over 18 hours and then left at 0° C. for 2 hours. The solid was collected by filtration, washed with toluene (2×0.5 ml) and dried in vacuo at 50° C. to give the title conpound (205 mg) as crystals.

Examples of Properties of a sodium salt of (−)2-[[2-(4-hydroxyphenyl)ethyl]thio}-3-[4-(2-{4-[(methylsulfonyl)oxy]-phenoxy )ethyl)phenyl]propanoic acid DSC showed an endotherm with an extrapolated onset ten,rature of 155° C. TGA showed a weight loss of 0.3% w/w between 25-110° C. and 0.7% w/w between 110-165° C. DSC analysis repeated on purer sample Iiiay give a higher melting point. Crystals of the sodium salt of (S)-2-{[2-(4-hydroxyphenyl)ethyl]thio}-3-[4-(2-{4-[(methylsulfonyl)oxy]-phenoxy}ethyl)phenyl]propanoic acid (obtained by way of the example above and/or by other ways) were analyzed by XRPD and the results are tabulated below and are shown m Figure B. Figure B.XRPD pattern of sodium salt of (−)-2-{[2-(4-hydroxyphenyl)ethyl]thio}-3-[4-(2-{4-[(methylsulfonyl)oxy]-phenoxy}ethyl)phenyl]propanoic acid

[image]

| d-value | intensity |
|---|---|
| (Angstrom) | (rel) |
| 15.9 | w |
| 12.8 | s |
| 11.8 | m |
| 8.2 | m |
| 6.5 | w |
| 5.7 | m |
| 5.5 | m |
| 5.3 | m |
| 4.91 | m |
| 4.74 | m |
| 4.68 | m |
| 4.30 | m |
| 4.16 | s |
| 4.08 | m |
| 4.01 | w |
| 3.78 | w |
| 3.73 | w |
| 3.55 | w |
| 3.43 | m |
| 2.89 | w |
| 2.77 | w |
| 2.65 | w |

Biological Activity

The activity of the conmounds of the invention is demonstrated in the assays described in WO03/051821.

1. A potassium salt or a sodium salt of (−)-2-{[2-(4-hydroxyphenyl)ethyl]thio )-3-[4-(2-{-[(methylsulfonyl}oxy]phenoxy}ethyl)phenyl]propanoic acid.

2. A salt according to claim 1 which is a potassium salt.

3. A salt according to claim 1 which is a sodium salt.

4. A salt as claimed in any one of claims 1 to 3 which may be a solvate, a hydrate, a mixed solvatehydrate, an ansolvate or an anhydrate.

5. A salt as claimed in any one of claims 1 to 4 in crystalline or partially crystalline form.

6. A pharmaceutical formulation comprising a compound according to any one of claims 1 to 5 in admixture with pharmaceutically acceptable adjuvants, diluents and/or carriers.

7. A method of treating or preventing lipid disorders (dyslipidemia) whether or not associated with insulin resistance comprising the adminlstratiou of a compound according to any one of claims 1 to 5 to a mamial in need thereof.

8. The use of a compound according to any one of claims 1 to 5 in the manufacture of a medicament for the treatment of lipid disorders (dyslipidemia) whether or not associated with insulin resistance.

9. A method of treating or preventing type 2 diabetes comprising the administration of an effective amount of a compound according to any one of claims 1 to 5 to a mammal in need thereof.

10. A pharmaceutical composition comprising a compound according to any one of claims 1 to 5 combined with another therapeutic agent that is useful in the treatment of disorders associated with the developinent and progress of atherosclerosis such as hypertension, hyperlipidaemias, dyslipidaemias, diabetes and obesity.
